(19) United States
(12) Patent Application Publication
(10) Pub. No.: US 2026/0110652 A1
(43) Pub. Date: Apr. 23, 2026

(54) ELECTRONIC NOSE WITH GAS EXCHANGE SYSTEM

(71) Applicant: Ainos, Inc., San Diego, CA (US)

(72) Inventors: Chia-Nan LIAO, Zhubei City (TW); Chia-Pin HUANG, Zhubei City (TW); Chun-Hsien TSAI, Zhubei City (TW)

(21) Appl. No.: 19/197,062

(22) Filed: May 2, 2025

(30) Foreign Application Priority Data
Oct. 22, 2024 (TW) 113140182

(51) Int. Cl. G01N 27/12; G01N 1/22

(52) U.S. Cl. CPC G01N 27/12; G01N 1/2273; G01N 2001/2288

(57) ABSTRACT

An electronic nose for rapid detection comprises a gas intake unit, a detection unit, an evacuation unit, and a processing unit. The gas intake unit includes a first and a second channel. The electronic nose is configured to allow external gas to enter the chamber through the second channel periodically at a time interval and intake of the external gas lasts for a detection time period. The periodical intakes are repeated until a detection signal satisfies a criterion. Once the criterion is satisfied, the external gas is introduced into the chamber through the first channel via a filter continuing until a detection signal obtained by a gas sensor device and environmental parameters obtained by one or more environmental sensor devices reach an equilibrium. Then, the external gas is introduced into the chamber through the second channel without filtering. Subsequently, gas-related information related to the external gas is obtained based on the detection signal.

### FIELD OF THE INVENTION

The present invention relates to an electronic nose, particularly an electronic nose capable of rapid detection and suitable for integration into robots.

### BACKGROUND OF THE INVENTION

Robots are widely applied in modern society in various fields including factory automation, home care, environmental exploration, disaster rescue, security patrolling, and gas detection. Typically, robots are equipped with a variety of sensing devices to detect their surroundings and take appropriate measures. Among these sensing devices, an electronic nose is capable of distinguishing and quantifying both simple and complex odors. It uses gas sensors to detect gases in the environment, performs comparisons and analyses, and thereby realizes multiple functions. For example, it can detect harmful gases and issue alerts, monitor air quality, detect dangerous conditions such as fires or gas leaks, be applied in disease and public health, or be used for food analysis.

In existing electronic nose technologies, continuous monitoring of the environment requires repeated calibration of gas sensors and execution of gas identification processes. This occurs regardless of whether the environmental gas changes, resulting in time-consuming identification procedures that fail to reflect real-time gas variations. Additionally, constantly performing these identification processes consumes significant power and reduces the operational lifespan of the electronic nose.

### SUMMARY OF THE INVENTION

In at least one example of the present disclosure, an electronic nose equipped with a gas exchange system is provided to analyze external gas. The electronic nose includes a gas intake unit, a detection unit, an evacuation unit, and a processing unit. The gas intake unit includes a filter, a first gas intake channel and a second gas intake channel, with the filter connected to the first gas intake channel to allow fluid communication. The detection unit includes a chamber and a detection module, where the chamber is in fluid communication with both the first and second gas intake channels. The detection module includes a gas sensor device and one or more environmental sensor devices. The gas sensor device detects gas within the chamber and generates a detection signal in response to the gas, while the environmental sensor devices measure environmental parameters inside the chamber. The evacuation unit is connected to the chamber, and the processing unit is connected to the detection module to receive the detection signal it produces.

The electronic nose is configured to perform the following steps:
Step 1: following each standby time period, allowing external gas to enter the chamber via the second gas intake channel for a detection time period, wherein the detection time period is shorter than the standby time period;
Step 2: repeating Step 1 until the detection signal meets a criterion;
Step 3: when the criterion is met, allowing the external gas, after being filtered by the filter, to enter the chamber via the first gas intake channel continuously until the detection signal and the environmental parameters reach an equilibrium, after which the first gas intake channel is closed; and
Step 4: allowing the external gas to enter the chamber via the second gas intake channel and obtaining gas-related information related to the external gas based on the detection signal generated based on the external gas entering the chamber from the second gas intake channel.

### BRIEF DESCRIPTION OF THE DRAWINGS

Example embodiments of the present disclosure will be described with reference to the accompanying drawings briefly described below.

FIG. 1 is a schematic diagram of a robot according to an embodiment of the present invention.

FIG. 2A is a schematic block diagram illustration of an electronic nose, according to various exemplary embodiments.

FIG. 2B is a schematic block diagram illustration of an electronic nose, according to various exemplary embodiments.

FIG. 3 is a schematic block diagram illustration of an electronic nose, according to other exemplary embodiments.

FIG. 4 is a schematic flow-chart illustration of a method for operation of an electronic nose, according to various exemplary embodiments.

FIG. 5 shows resistance variation of the detection signal in a monitoring mode, according to various exemplary embodiments.

FIG. 6 shows resistance variation of the detection signal in an identification mode, according to various exemplary embodiments.

### DETAILED DESCRIPTION

It should be understood that the terminology used in the description of various embodiments is for illustration only and is not intended to be limiting. Unless otherwise explicitly stated by context or the number of components is deliberately restricted, the singular terms such as “a” or “the” also include plural forms. Furthermore, the terms “including” and “comprising” indicate the presence of the stated features, components, and/or assemblies without excluding the addition or presence of one or more other features, components, assemblies, or their combinations. Indefinite and definite articles are intended to include both singular and plural meanings unless the context clearly indicates otherwise.

The present invention discloses an electronic nose. In one embodiment, the electronic nose is suitable for mounting on a robot, which may be an autonomous mobile robot, an automated guided vehicle, an articulated robot, a humanoid robot, a collaborative robot, or a hybrid robot. It may also be a mechanical robot or a bionic robot. Nonlimiting examples include patrolling robots, exploration robots, and home care robots. Although examples are provided, the term “robot”is to be interpreted broadly.

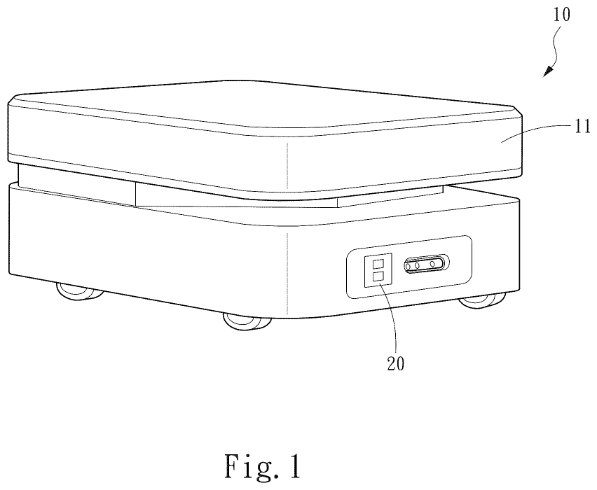

FIG. 1 illustrates a robot according to an embodiment of the present invention. The robot 10 is a wheeled robot equipped with an electronic nose 20. The robot 10 includes a robot body 11, on which the electronic nose 20 is mounted. At least a portion of the electronic nose 20 is exposed from a casing of the robot 10 so as to be exposed to atmospheric/ambient environment for real-time detection. With the electronic nose 20, the robot 10 can continuously monitor changes in the surrounding gas environment and take necessary actions based on the detection results. In the present disclosure, “external gas” refers to the ambient gas in the space where the robot 10 or the electronic nose 20 is located.

For example, in factory or home environments, there may be excessive amounts of harmful gases such as carbon monoxide, carbon dioxide, ozone, sulfur dioxide, nitrogen dioxide, volatile organic compounds, formaldehyde, and the like. The robot 10 may use the electronic nose 20 to detect whether these harmful gases are present and whether their concentrations exceed a safety standard, thereby generating alerts or activating a ventilation system to enhance gas exchange with the external environment. Alternatively, in unknown or extreme environments such as deep-sea, caves, or space, a mobile robot 10 equipped with the electronic nose 20 can perform real-time analysis of the gas composition in the environment.

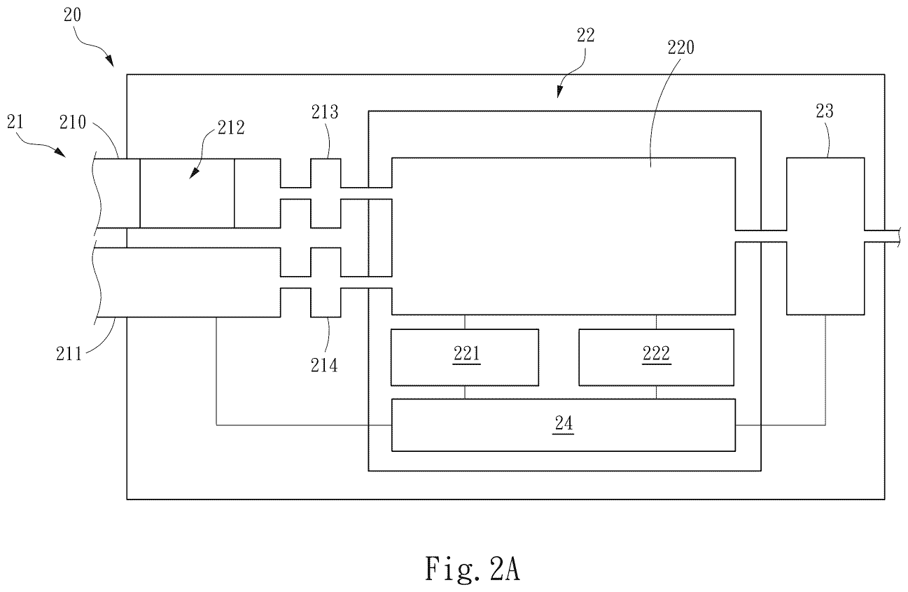

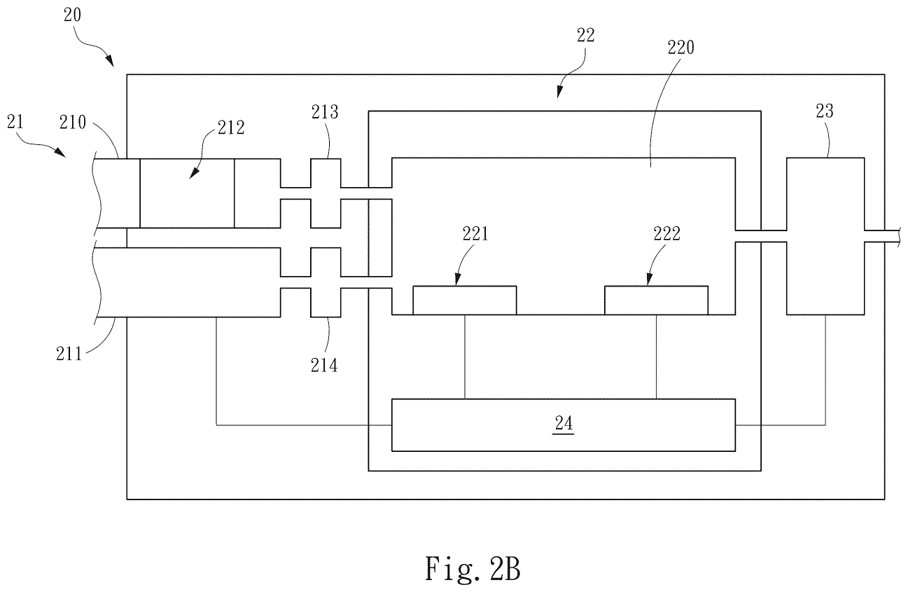

Referring to FIG. 2A, in one embodiment the electronic nose 20 comprises a gas intake unit 21, a detection unit 22, an evacuation unit 23, and a processing unit 24. The gas intake unit 21 has an intake port that is in communication with the external environment; in one embodiment, this intake port is provided on the outer casing of the robot 10 so that external gas may enter the electronic nose 20. The detection unit 22 comprises a chamber 220 and a detection module. The detection module may be located inside the chamber 220. In other examples, the detection module may be located at another position as long as it is capable of detecting the gas within the chamber 220. For example, the detection module may be at least partially exposed to the chamber 220. The chamber 220 includes one or more gas intake ports and a discharge port. The upstream portion of the chamber 220 is fluidly connected to the gas intake unit 21 via an intake port to receive external gas, while the downstream portion is fluidly connected to the evacuation unit 23, which uses the negative pressure generated by the evacuation unit 23 to assist in discharging the gas. The evacuation unit 23 may be a pump.

The processing unit 24 is connected to both the gas intake unit 21 and the evacuation unit 23 to control the channels and flow rate. The gas intake unit 21 is configured to selectively introduce either filtered external gas (clean gas) or unfiltered external gas (the gas to be detected), with the processing unit 24 controlling which type of gas is allowed to enter the chamber 220 as well as controlling the operation of the evacuation unit 23 and the magnitude of the negative pressure to adjust the flow rate of the gas entering or passing through the chamber 220.

In one embodiment, the gas intake unit 21 may comprise two gas pipelines. For example, the gas intake unit 21 may include a first gas intake channel 210 and a second gas intake channel 211, with a filter 212 arranged within or before or after the first gas intake channel 210. The first gas intake channel 210 introduces the filtered external gas into the chamber 220, while the second gas intake channel 211 is used to introduce unfiltered external gas. The external gas enters the chamber 220 without undergoing any filtering or processing.

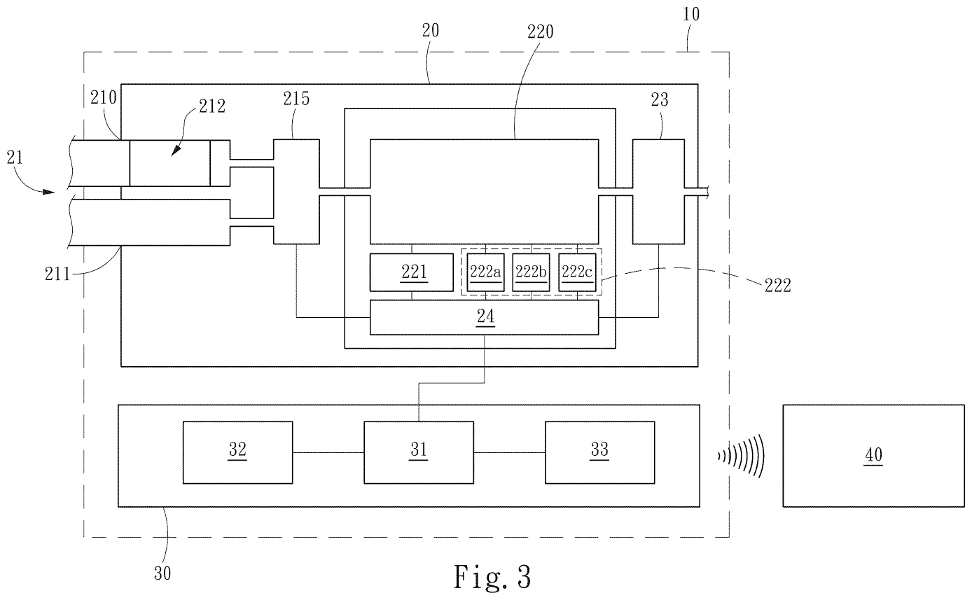

In one embodiment, the first gas intake channel 210 and the second gas intake channel 211 are each provided with a first valve 213 and a second valve 214 that are connected with and controlled by the processing unit 24. Alternatively, in another embodiment, the electronic nose 20 may include a three-way valve 215 as shown in FIG. 3, which is connected to and controlled by the processing unit 24 and is disposed downstream of the first intake channel 210 and the second gas intake channel 211. This three-way valve 215 selectively connects either the first gas intake channel 210 or the second gas intake channel 211 with the chamber 220. In one embodiment, the filter 212 may be an activated carbon filter element used to adsorb or filter volatile organic compounds (VOCs), although the present invention is not limited to this.

Returning to FIG. 2A, the detection module includes a gas sensor device 221 and one or more environmental sensor devices 222. The gas sensor device 221 and the environmental sensor devices 222 may be arranged within the chamber 220 or at least partially exposed in the chamber 220 as shown in FIG. 2B, but are not limited to this arrangement; the detection module may be located at any position that can contact and detect the gas in the chamber 220. In one embodiment, the gas sensor device 221 is a device that generates or varies an electrical signal in response to gas, such as a chemical resistance-type or electrochemical gas sensor array or a semiconductor gas detector. The present invention is not limited to these examples, the gas sensor device 221 may also be implemented using other forms or structures, such as optical or electrochemical gas sensors.

The gas sensor device 221 is configured to detect gas or changes in gas within the chamber 220. This may involve detecting the type of gas present, the existence of one or more specific components, the concentration or quantity of a specific component (or whether it reaches a certain value), whether the gas in the chamber 220 conforms to a specific composition, or changes in specific components, composition, or concentration. Specific components may include oxygen, carbon monoxide, hydrogen sulfide, ammonia, chlorine, ozone, sulfur dioxide, nitrogen dioxide, natural gas, liquefied petroleum gas, methane, or propane. Specific compositions may include toxic or combustible gases.

The gas sensor device 221 detects the gas within the chamber 220 and produces a detection signal that is responsive to the gas present. For instance, if a chemical resistance-type sensor array is used, the detection signal may be represented as a resistance value (for example, changing from zero to a certain value) or as a change in resistance (for example, from an initial value R0 to a subsequent value). The detection signal may then be used to derive gas-related information related to the external gas. The gas-related information may indicate the presence of one or more specific components in the external gas, the concentration or quantity of the specific component (or whether it reaches a certain value), whether the external gas conforms to a specific composition, or changes in the specific components, composition, or concentration of the external gas.

The environmental sensor devices 222 are used to detect one or more environmental parameters within the chamber 220, which may include temperature, humidity, atmospheric pressure, or any combination thereof. As shown in FIG. 3, depending on the environmental parameters to be detected, the environmental sensor devices 222 may include a temperature sensor 222a, a humidity sensor 222b, and a pressure sensor 222c. The temperature sensor 222a, humidity sensor 222b, and pressure sensor 222c measure the temperature, humidity, and pressure in the chamber 220, respectively.

In the embodiment shown in FIG. 3, the electronic nose 20 is mounted on the robot 10 in a modular manner, and the processing unit 24 may be further connected to a control unit 30 of the robot 10. The control unit 30 may comprise a processor 31, a database 32, and a transmission interface 33 and may be used to control the processing unit 24, receive signals from it, or serve as a communication path between the electronic nose 20 and other external components. In one embodiment, the processor 31 receives and processes the detection signal from the gas sensor device 221 and the environmental sensor devices 222. For example, it may compare the detection signal with data stored in the database 32 and generate an analysis result related to the external gas. In one embodiment, the processor 31 may perform artificial intelligence computations, enabling the robot 10 to conduct generative AI processing on the detection signal and/or the environmental parameters locally. In other embodiments, the control unit 30 may be connected via the transmission interface 33 to an external device 40, such as a server or an external database. The transmission interface 33 may support wired or wireless communication protocols such as WiFi, BLE, Bluetooth, Z-Wave, USB, or Zigbee. It should be understood that in other embodiments, the control unit 30 may be integrated with the electronic nose 20 as a single module and is not limited to the configurations described above.

In the detection process of the electronic nose 20, one of the primary sources of electrical power consumption is the operation of the evacuation unit 23. If the evacuation unit 23 remains active whenever the electronic nose 20 is operational, continuously drawing external gas into the chamber 220, it results in high electrical power consumption. This shortens the operational duration of the electronic nose 20 and reduces the lifespan of the evacuation unit 23. To address this, the present invention proposes operating the electronic nose 20 in either a monitoring mode or an identification mode. The monitoring mode can be regarded as a phase with lower detection accuracy but reduced electrical power consumption, while the identification mode is a phase with higher detection accuracy and greater electrical power consumption. In one example, the electronic nose 20 operates normally in the monitoring mode by default and remains in this mode continuously until a designated condition is met, at which point the electronic nose 20 switches to the identification mode. In the example, the designated condition may be a change of the external gas requiring further determination or analyzed.

The monitoring mode involves multiple cycles of a standby time period and short-duration gas intake. The monitoring mode continues until the detection signal during the short-duration gas intake meets a criterion, triggering a switch from the monitoring mode to the identification mode. The identification mode involves a single cycle of a long-duration gas intake followed by a detection. It should be understood that the term “short-duration” is relative to “long-duration,” and within these modes, the time periods may be the same or different. For example, the standby time is greater than the duration of the short-duration gas intake. By adjusting the ratio of the short-duration to the long-duration periods (or periods of the short-duration and the long-duration), as well as the ratio of the standby time to the duration of the short-duration gas intake (or periods of the standby time period and the short-duration gas intake), the operational time of the evacuation unit 23 during the overall detection process of the electronic nose 20 can be significantly reduced. This not only saves electrical power consumption but also extends the lifespan of the evacuation unit 23.

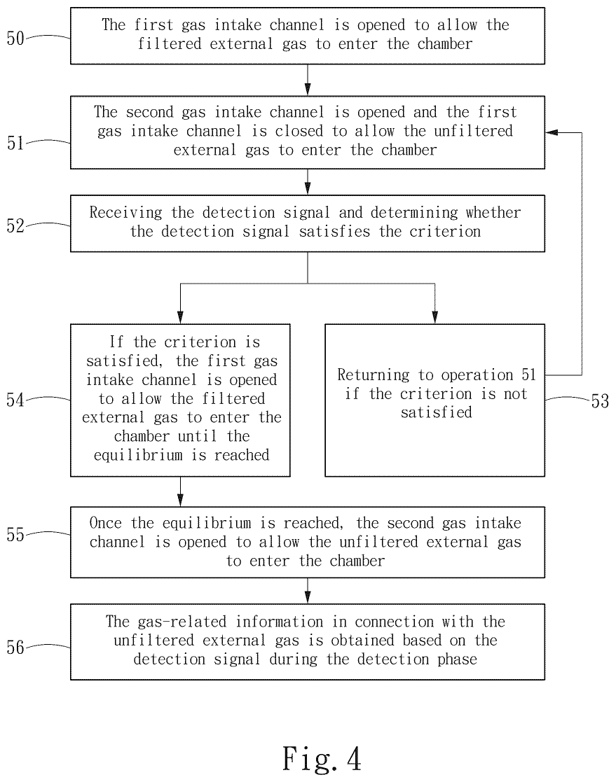

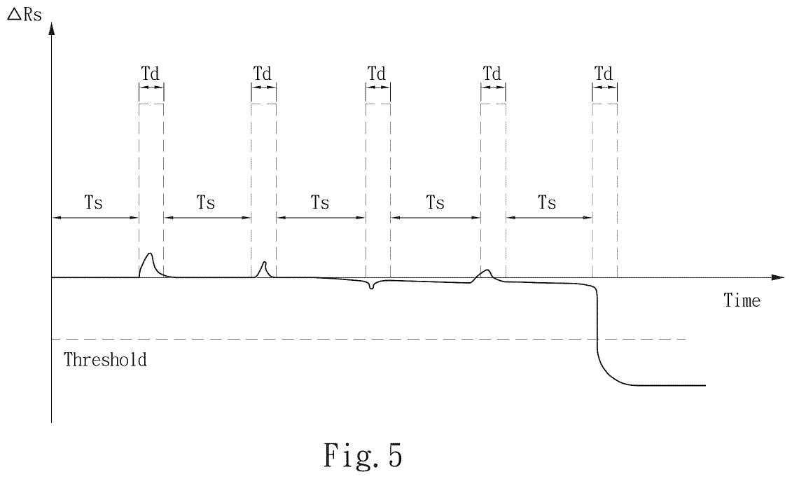

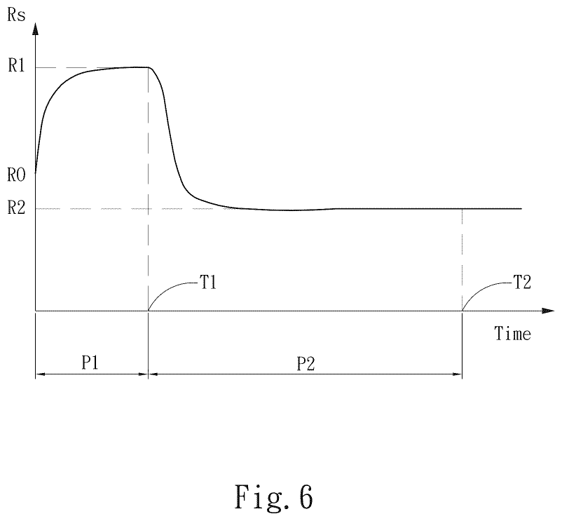

In the following example, the gas sensor device 221 is a chemical resistance-type gas sensor, and the detection signal is a resistance value. Referring to FIG. 4, which illustrates the operational flow of the electronic nose 20, along with FIGS. 5 and 6, the electronic nose 20 normally operates in the monitoring mode by default and switches to the identification mode only when the criterion is met. In this example, before entering the monitoring mode, the evacuation unit 23 is activated, and the first gas intake channel 210 is opened (with the second gas intake channel 211 closed), allowing filtered external gas (filtered gas) to enter the chamber 220 (Operation 50) to clean the chamber 220. Subsequently, the electronic nose 20 enters the monitoring mode, cycling through periods of standby time period and short-duration gas intake. During the standby time, the evacuation unit 23 is not activated, so no gas enters the chamber 220, and no detection occurs. During the short-duration gas intake, the evacuation unit 23 is activated, with the first gas intake channel 210 closed and the second gas intake channel 211 opened, allowing unfiltered external gas (the gas to be detected) to enter the chamber 220 from the second gas intake channel 211 (Operation 51). This gas is then detected by the gas sensor device 221 (Operation 52).

FIG. 5 illustrates the change in the resistance value (ΔRs) of the gas sensor device 221 over time during the monitoring mode, where ΔRs=R_{S(t)}−R_{S(t-1}). Here, R_{S(t)} represents the resistance value at time t, and R_{S(t-1)} represents the resistance value at the previous time point t−1, with the time interval between t and t−1 chosen according to requirements (for instance, t could represent the 5th second while t−1 might represent the 3rd second). The monitoring mode includes multiple standby time periods Ts (the standby time) and multiple detection time periods Td (the short-duration gas intake), with the detection time period Td following the standby time period Ts. During the standby time period Ts, the evacuation unit 23 does not draw gas (the evacuation unit 23 is offline), and the evacuation unit 23 only operates during the shorter detection time period Td. In other words, during the standby time period Ts, since no external gas is introduced into the chamber 220, the resistance value Rs undergoes only negligible variation, as illustrated in the five Ts segments in FIG. 5. During the detection time period Td, external gas is introduced into the chamber 220, causing the resistance value to change, as shown in the five Td segments in FIG. 5. The standby time period is designed to reduce the power consumption of the evacuation unit 23, and any change in the detection signal during the standby time period may be considered meaningless. In other words, no further action is taken even if a change in the detection signal occurs during the standby time period. These cycles of standby and detection time periods continue uninterrupted.

The detection signal is evaluated to determine if the detection signal meets a criterion. In this example, the criterion is defined as a change in the resistance value exceeding a threshold. The change may refer to either the rate of change of the resistance value or an increment or decrement in the resistance value. If the change in resistance value (ΔRs) induced by the external gas is small or below the threshold, the electronic nose 20 continues operating in the monitoring mode (Operation 53), as shown in the first four Td segments in FIG. 5. However, if the change in resistance value ΔRs induced by the external gas is significant or exceeds the threshold, the electronic nose 20 switches to the identification mode, as shown in the fifth Td segment in FIG. 5 (Operation 54). In one example, the detection time period Td is shorter than the standby time period Ts. In one example, the ratio of the detection time period Td to the standby time period Ts is between 0 and 1, such as less than ⅕, 1/10, or 1/15.

FIG. 6 illustrates the variation of the resistance value (Rs) over time in the identification mode. The identification mode includes two phases: a pre-detection phase P1 and a detection phase P2. In the pre-detection phase P1, the processing unit 24 activates the evacuation unit 23 and controls the gas intake unit 21 to allow filtered external gas (clean gas) to enter the chamber 220 (Operation 54). The filtered gas is not the gas to be detected, it may be regarded as background, reference, or cleaning gas used to bring the chamber 220 to an equilibrium prior to detection. In some aspects, the pre-detection phase P1 may also be considered a cleaning stage.

During this phase, the processing unit 24 receives the detection signal from the gas sensor device 221 and the environmental sensor devices 222 and monitors whether the values have reached an equilibrium (Operation 54). The equilibrium may be defined as the condition in which the detection signal and one or more environmental parameters in the chamber 220 have reached a steady value or slightly varied within a range. The equilibrium may be a state that the balanced detection signal and the balanced environmental parameters are consistently sustained under continuous gas flow conditions. The environmental parameters may include temperature, humidity, and atmospheric pressure. The equilibrium may involve a single environmental parameter (such as temperature only) or several parameters simultaneously; however, the greater the number of environmental parameters that reach the equilibrium, the more effectively the detection process can proceed.

“Reaching the equilibrium” means that the detection signal and the environmental parameters in the chamber 220 remain substantially constant over time (for example, varying within ±10%, ±5%, or ±1% of a certain value). The processing unit 24 may determine that the equilibrium has been reached if the detection signal and the environmental parameters remain continuously and substantially constant for a predetermined threshold period during the pre-detection phase P1. In one embodiment, the evacuation unit 23 is controlled so that the flow rate of the filtered gas entering the chamber 220 remains substantially constant over time, thereby ensuring a stable gas flow within the chamber 220 and facilitating rapid attainment of the equilibrium.

As shown in FIG. 6, during the pre-detection phase P1 the resistance value of the detection signal generated by the gas sensor device 221 gradually increases from an initial resistance R0 and stabilizes at a first resistance value R1 at time T1. At this point, the equilibrium is achieved. The time from the start until T1 is defined as a first time interval.

Once the equilibrium is reached, the electronic nose 20 proceeds to the detection phase P2 (Operation 55). In this embodiment, upon transitioning from the pre-detection phase P1 to the detection phase P2, the processing unit 24 maintains the operation of the evacuation unit 23 and controls the gas intake unit 21 to allow unfiltered external gas (the gas to be detected) to enter the chamber 220. In the detection phase P2, the gas within the chamber 220 is the unfiltered external gas to be detected. In this example, the gas intake unit 21 operates continuously, with the first gas intake channel 210 open and the second gas intake channel 211 closed during the pre-detection phase P1, and the second gas intake channel 211 opened and the first gas intake channel 210 closed during the detection phase P2.

When different gas is introduced into the chamber 220, as shown in FIG. 6, the resistance value of the detection signal from the gas sensor device 221 shifts from R1 to R2, stabilizing at T2 and reflecting one or more properties of the unfiltered external gas. The period from the end of T1 (or the start of T2) to the end of T2 is defined as a second time interval. The processing unit 24 receives the detection signal from the gas sensor device 221 and the environmental sensor devices 222 (Operation 56) and processes them to derive gas-related information based on the detection signal. In one embodiment, this detection occurs at room temperature without additional heating of the gas in the chamber 220. However, the present invention is not limited to this, as in some embodiments the detection may be carried out with the gas in the chamber 220 heated, e.g., to temperatures above 50° C. or between 50° C. and 450° C.

The gas entering chamber 220 during the first time interval is a filtered gas to clean the chamber, while the gas entering during the second time interval is the gas to be detected. The evacuation unit 23 operates continuously, without deactivation, throughout both the first and second time intervals. As a result, gas flows uninterrupted into the chamber 220 across both time periods (the first and second time intervals). Specifically, during the first time interval, the first gas intake channel 210 is open while the second gas intake channel 211 is closed. During the second time interval, which follows immediately after the first time interval without any interruption, the first gas intake channel 210 is closed, and the second gas intake channel 211 is opened.

In the example, the gas-related information in connection with the unfiltered external gas is generated based on the detection signal during the detection phase P2. That is, the gas-related information is irrelevant with the detection signal of the filtered external gas during the pre-detection phase P1. In other words, the gas-related information is independent of the detection signal during the pre-detection phase P1. The detection signal during the pre-detection phase P1 is used solely to determine whether equilibrium has been reached.

In some example, the detection signal during the detection phase P2 may be compared with data stored in the database 32 and generate an analysis result related to the external gas.

In one embodiment, the equilibrium is in a dynamic mode, which involves two aspects. First, the gas in the chamber 220 is in motion, meaning the gas flows rather than is static. Second, during the pre-detection phase P1, the detection signal and the environmental parameters remain substantially constant for the predetermined threshold period under this flowing gas condition. In one embodiment, when transitioning from the pre-detection phase P1 to the detection phase P2, the evacuation unit 23 remains operational, and the external gas is continuously introduced into the chamber 220, initially through the first gas intake channel 210 and then through the second gas intake channel 211.

In addition, the filtered external gas within the chamber 220 generates a first gas flow and the unfiltered external gas produces a second gas flow, with both flows maintaining substantially the same flow rate during the pre-detection phase P1 and the detection phase P2, respectively. Because the evacuation unit 23 is not turned off during the transition, the gas flow in the chamber 220 remains continuous, with only the gas changing. Consequently, the variation in environmental parameters within the chamber 220 is minimized, meaning that the equilibrium is less disturbed or disrupted, and does not need to be re-established, thereby improving detection accuracy and reducing time consumption. Under these conditions, the equilibrium may be interpreted as a dynamic equilibrium.

The present invention recognizes that when introducing external gas for detection, it is essential to maintain a stable detection environment, that is, to achieve equilibrium, in order to obtain accurate detection results. Therefore, before introducing the unfiltered gas to be detected, the electronic nose 20 first maintains the chamber 220 in a dynamic equilibrium in which, despite continuous gas flow, the one or more environmental parameters remain substantially constant, and then, without interrupting the gas flow, switches to introducing the unfiltered gas. This ensures that the flow rate remains substantially identical, meaning that the gas pressure within the chamber 220 remains substantially the same during both phases.

However, in certain aspects of the present invention, the equilibrium is not necessarily dynamic; it may also be static. In the static mode, after the pre-detection phase P1 is completed, the evacuation unit 23 is turned off, allowing the detection signal and environmental parameters in the chamber 220 to reach the equilibrium in the absence of gas flow before entering the detection phase P2. Accordingly, the electronic nose 20 may be selectively operated in either a dynamic or static equilibrium mode.

The electronic nose 20 of the present invention is designed to operate in the monitoring mode by default, entering the identification mode only when the criterion are met. In the monitoring mode, the operational time of the evacuation unit 23 significantly exceeds its downtime, substantially reducing power consumption and extending the lifespan of the evacuation unit 23.

What is claimed is:

1. An electronic nose with a gas exchange system, comprising:
a gas intake unit, including a filter, a first gas intake channel, and a second gas intake channel, wherein the filter is in fluid communication with the first gas intake channel;
a detection unit, including a chamber and a detection module, wherein the chamber is in fluid communication with the first and second gas intake channels, and the detection module comprises a gas sensor device and one or more environmental sensor devices, the gas sensor device being configured to detect gas in the chamber and generate a detection signal responsive to the gas in the chamber, and the environmental sensor devices being configured to detect one or more environmental parameters in the chamber;
an evacuation unit connected to the chamber; and
a processing unit connected to the detection module and configured to receive the detection signal generated by the detection module;
wherein the electronic nose is configured to perform the following steps:
Step 1: following each standby time period, allowing an external gas to enter the chamber via the second gas intake channel for a detection time period, wherein the detection time period is shorter than the standby time period;
Step 2: repeating Step 1 until the detection signal meets a criterion;
Step 3: when the criterion is met, allowing the external gas, after being filtered by the filter, to enter the chamber via the first gas intake channel continuously until the detection signal and the environmental parameters reach an equilibrium, after which the first gas intake channel is closed; and
Step 4: allowing the external gas to enter the chamber via the second gas intake channel and obtaining gas-related information related to the external gas based on the detection signal generated based on the external gas entering the chamber from the second gas intake channel;
wherein the criterion is that a change in the detection signal generated by the gas sensor device during the detection time period reaches a threshold.

2. The electronic nose according to claim 1, wherein the processing unit is connected to a control unit, the control unit comprising a processor and a database, the processor being configured to perform artificial intelligence computations based on the database and the detection signal to generate an analysis result.

3. The electronic nose according to claim 1, wherein the evacuation unit is deactivated during the standby time period and activated during the detection time period.

4. The electronic nose according to claim 1, wherein the standby time period and the detection time period alternate continuously, with one immediately following the other without any interruption.

5. The electronic nose according to claim 1, wherein any change in the detection signal during the standby time is considered meaningless.

6. The electronic nose according to claim 1, wherein the equilibrium is defined as a condition in which, over a time interval, the detection signal and the environmental parameters each continuously remain substantially constant.

7. The electronic nose according to claim 1, wherein the environmental parameters include the chamber's temperature, humidity, atmospheric pressure, or any combination thereof.

8. The electronic nose according to claim 1, wherein the gas sensor device is a chemical resistance-type gas sensor and the detection signal is a resistance value.

9. The electronic nose according to claim 1, wherein the environmental sensor devices include a temperature sensor, a humidity sensor, a pressure sensor, or any combination thereof.

10. The electronic nose according to claim 1, wherein, upon reaching the equilibrium and when introducing the external gas from the second gas intake channel, the environmental parameters remain substantially constant.

11. The electronic nose according to claim 1, wherein the external gas enters the chamber through the second gas intake channel is without filtering.

12. The electronic nose according to claim 1, wherein the gas-related information is irrelevant with the detection signal of the filtered external gas.

13. The electronic nose according to claim 1, wherein the external gas enters the chamber via the first gas intake channel through the filter continuously during a first time interval and the external gas enters the chamber via the second gas intake channel continuously during a second time interval, and wherein the equilibrium is a period within the first time interval during which the detection signal and the environmental parameters each continuously maintain substantial constancy.

14. The electronic nose according to claim 13, wherein the gas entered the chamber throughout the first time interval is a filtered gas to clean the chamber, and the gas entered the chamber throughout the second time interval is a gas to be detected.

15. The electronic nose according to claim 13, wherein the evacuation unit is configured to operate continuously without being deactivated throughout the first time interval and the second time interval, with the second time interval immediately succeeding the first time interval.

16. The electronic nose according to claim 13, wherein the gas continuously flows into the chamber throughout the first time interval and the second time interval, with the second time interval immediately succeeding the first time interval.

17. The electronic nose according to claim 13, wherein during the first time interval, the first gas intake channel is configured to be opened and the second gas intake channel is configured to be closed, and wherein during the second time interval, the first gas intake channel is configured to be closed and the second gas intake channel is configured to be opened.

18. The electronic nose according to claim 13, wherein the second time interval immediately follows the first time interval without interruption.
